(19) United States
(12) Patent Application Publication
(10) Pub. No.: US 2003/0212109 A1
(43) Pub. Date: Nov. 13, 2003

(54) Pharmaceutical compositions and their use

(76) Inventors: Stephen Nicholas Mitchell, Hampshire (GB); Ian Alexander Pullar, Hampshire (GB)

Correspondence Address: ELI LILLY AND COMPANY, PATENT DIVISION, P.O. BOX 6288, INDIANAPOLIS, IN, 46206-6288, US

(21) Appl. No.: 10/276,107

(22) PCT Filed: May 21, 2001
(86) PCT No.: PCT/US01/10824

(30) Foreign Application Priority Data
Jun. 2, 2000 (GB) 0013503.8

(51) Int. Cl. A61K 31/454; A61K 31/496; A61K 31/137

(52) U.S. Cl. 514/323; 514/254.09; 514/651

(57) ABSTRACT

A pharmaceutical composition comprising a serotonin transport inhibitor and a 5-HT1D antagonist, together with a pharmaceutically acceptable diluent or carrier.

This invention relates to pharmaceutical compositions and their uses.

It is well known that serotonin plays an important role in the central nervous system, and many disorders of the central nervous system can be attributed to an imbalance of this and other, similar, neurotransmitters.

Certain compounds that are modulators of serotonin are disclosed in European Patent Application 0 780 388 where they are described as active at the serotonin, 5-HT_{1D}, receptor. Such compounds are examples of agents that are 5-HT_{1D} antagonists.

A further and well known group of compounds that affect concentrations of serotonin in the synapse are inhibitors of the serotonin transporter such as serotonin re-uptake inhibitors (SRI's), for example fluoxetine, duloxetine, venlafaxine, milnacipran, citalopram, fluvoxamine, paroxetine, sertraline and nefazodone. One problem associated with such SRI's is the delay that occurs in the onset of their activity in patients.

It has now been found, preclinically, that 5-HT_{1D} antagonists enhance the efficacy of serotonin transport inhibitors—an effect that may result in a more rapid onset of clinical activity.

Thus, the invention provides a pharmaceutical composition comprising a serotonin transport inhibitor and a 5-HT_{1D} antagonist.

More particularly, the invention provides a pharmaceutical composition comprising an inhibitor of the serotonin transporter and a compound of the formula:

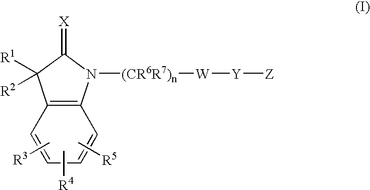

in which

R¹ and R² are each hydrogen, C₁₋₄ alkyl, C₁₋₄ alkoxy, HO—C₁₋₄ alkyl, C₁₋₄ alkoxy-C₁₋₄ alkyl, C₁₋₄ alkylthio, halo, Ph, PhCR′R″— where Ph is optionally substituted phenyl and R′ and R″ are each hydrogen or C₁₋₄ alkyl, or R¹ and R² together with the carbon atom to which they are attached form a C₃-₆ cycloalkyl group, >C═O, >C═NOR′ where R′ is hydrogen or C₁₋₄ alkyl,

R³, R⁴ and R⁵ are each hydrogen, halo, nitro, C₁₋₄ alkyl, C₁₋₄ alkoxy, C₁₋₄ alkylthio, C₁₋₄ alkyl-CO—, C₁₋₄ alkyl-S(O)ₘ— where m is 0, 1 or 2, R′R″N—SO₂—, —COOR′, —CONR′R″, —NR′R″, —N(OR′)COOR″, —COR′, —NHSO₂R′, where R′ and R″ are each hydrogen or C₁₋₄ alkyl,

R⁶ and R⁷ are each hydrogen or C₁₋₄ alkyl, and n is 1 to 6,

X is oxygen or sulphur,

W is

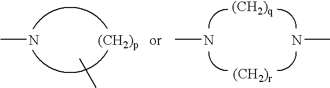

where p is 4 to 7, and q and r are each 1 to 3,

Y is

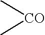

or —CH(OH)—, and

Z is optionally substituted phenyl or optionally substituted heteroaryl;

and salts and esters thereof.

In a further aspect of the invention, there is provided a method for treating a patient suffering from or susceptible to a disorder of the central nervous system, which comprises administering a combination of a serotonin transport inhibitor and a 5-HT_{1D} antagonist.

In another aspect of the invention there is provided a combination of an inhibitor of the serotonin transporter and a 5HT_{1D} antagonist, for use as a medicament.

In a further aspect of the invention there is provided the use of a combination of an inhibitor of the serotonin transporter and a 5HT_{1D} antagonist, for the manufacture of a medicament for the treatment of a disorder of the central nervous system.

In a still further aspect of the invention there is provided the use for the manufacture of a medicament for enhancing the action of a selective serotonin re-uptake inhibitor in increasing the availability of serotonin, of a combination of an inhibitor of the serotonin transporter and a 5-HT_{1D} antagonist.

In the above formula (I), a C₁₋₄ alkyl group includes methyl, ethyl, propyl, isopropyl, butyl and tert butyl, and is preferably methyl or ethyl. A C₁₋₄ alkoxy group is one such alkyl group linked to a ring via an oxygen atom, and a halo atom is preferably chlorine, bromine or fluorine, and especially chlorine or fluorine. A substituted phenyl group is phenyl substituted with one or more, for example one to three, substituents selected from, for example C₁₋₄ alkyl, especially methyl, C₁₋₄ alkoxy, especially methoxy and ethoxy, hydroxy, nitro, cyano, halo, especially chloro or fluoro, trihalomethyl, especially trifluoromethyl, carboxy and C₁₋₄ alkoxy-carbonyl.

A heteroaryl group can have one or more hetero atoms selected from, for example, oxygen, nitrogen and sulphur and preferably contains from 5 to 10 carbon atoms. Preferably a heteroaryl group contains a single hetero atom and is of the formula:

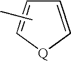

where Q is —O—, —S— or —NR—, and R is hydrogen or C₁₋₆ alkyl. Alternatively, a heteroaryl group can comprise a benzene fused ring as, for example:

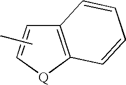

Further heteroaryl groups include those of the formula:

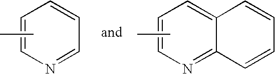

When n is greater than 1, the values of R⁶ and R⁷ need not be identical in each repeating methylene unit.

A substituted heteroaryl group is heteroaryl substituted with one or more, for example one or two, substituents selected from, for example, C₁-C₄ alkyl, especially methyl, and halo, especially chloro or fluoro.

A C₃₋₆ cycloalkyl group includes cyclopropyl, cyclobutyl, cyclopentyl and cyclohexyl.

Preferred compounds of formula (I) for use in the invention are those having one or more of the following features:

(i) X is oxygen

(ii) R¹ and R² are each hydrogen, C₁₋₄ alkyl, C₁₋₄ alkylthio or benzyl

(iii) R¹ and R² are both methyl

(iv) R¹ is hydrogen and R² is methyl

(v) R³, R⁴ and R⁵ are each hydrogen, halo or C₁₋₄ alkyl

(vi) R⁶ and R⁷ are both hydrogen

(vii) n is 2

(viii) W is

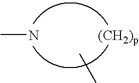

(ix) p is 5

(x) Y is

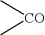

(xi) Z is optionally substituted phenyl

A preferred group of compounds for use in the invention is of the formula:

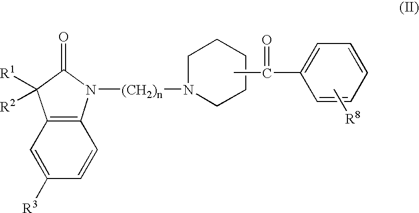

and preferably one in which R¹ and R² are each hydrogen or C₁₋₄ alkyl, R³ is hydrogen, C₁₋₄ alkyl or halo, n is 2, and R⁸ is hydrogen or halo. Preferably the benzoyl substituent is attached to the piperidinyl ring at the 4-position. A particularly preferred group is one in which R¹ and R² are both hydrogen and R³ is hydrogen or fluoro, n is 2 and R⁸ is halo, preferably fluoro; and salts thereof.

Compounds of formula (I) can exist in salt or ester form. Salts include the pharmaceutically acceptable, non-toxic addition salts with suitable acids, such as those with inorganic acids, for example hydrochloric, hydrobromic, nitric, sulphuric or phosphoric acids, or with organic acids, such as organic carboxylic acids, for example, glycollic, maleic, hydroxymaleic, fumaric, malic, tartaric, citric, salicyclic, o-acetoxybenzoic, or organic sulphonic, 2-hydroxyethane sulphonic, toluene-p-sulphonic, or naphthalene-2-sulphonic acid. A preferred salt form is the hydrochloride.

Compounds of formula (I) can also be utilised in ester form, such esters being aliphatic or aromatic. The most preferred esters are alkyl esters derived from C₁₋₄ alkanols, especially methyl and ethyl esters.

Examples of compounds of formula (I) and their pharmaceutically acceptable salts are:

3,3-Dimethyl-1-{2-[4-(4-fluorobenzoyl)-1-piperidinyl]-1-ethyl}-1,3-dihydro-2H-indol-2-one

1-{2-[4-(4-Fluorobenzoyl)-1-piperidinyl]-1-ethyl}-3-methyl-3-methylthio-1,3-dihydro-2H-indol-2-one

1-{2-[4-(4-Fluorobenzoyl)-1-piperidinyl]-1-ethyl}-3-methyl-1,3-dihydro-2H-indol-2-one

1-{2-[4-(4-Fluorobenzoyl)-1-piperidinyl]-1-ethyl}-3-methylthio-3-phenylmethyl-1,3-dihydro-2H-indol-2-one

1-{2-[4-(4-Fluorobenzoyl)-1-piperidinyl]-1-ethyl}-3-phenylmethyl-1,3-dihydro-2H-indol-2-one

1-{2-[4-(4-Fluorobenzoyl)-1-piperidinyl]-1-ethyl}-3-methyl-3-phenylmethyl-1,3-dihydro-2H-indol-2-one

3-Ethyl-1-{2-[4-(4-Fluorobenzoyl)-1-piperidinyl]-1-ethyl}-3-methyl-1,3-dihydro-2H-indol-2-one

1-{2-[4-(4-Fluorobenzoyl)-1-piperidinyl]-1-ethyl}-3-(1-methylethyl)-3-methylthio-1,3-dihydro-2H-indol-2-one

1-{2-[4-(4-Fluorobenzoyl)-1-piperidinyl]-1-ethyl}-3-(1-methylethyl)-1,3-dihydro-2H-indol-2-one

5-Bromo-3,3-dimethyl-1-{2-[4-(4-fluorobenzoyl)-1-piperidinyl]-1-ethyl}-1,3-dihydro-2H-indol-2-one

3,3-Dimethyl-1-{2-[4-(4-fluorobenzoyl)-1-piperidinyl]-1-ethyl}-5-methanesulfonyl-1,3-dihydro-2H-indol-2-one

3,3-Dimethyl-5-fluoro-1-{2-[4-(4-fluorobenzoyl)-1-piperidinyl]-1-ethyl}-1,3-dihydro-2H-indol-2-one

5,6-Difluro-3,3-dimethyl-1-{2-[4-(4-fluorobenzoyl)-1-piperidinyl]-1-ethyl}-1,3-dihydro-2H-indol-2-one

3,3-Dimethyl-1-{2-[4-(4-fluorobenzoyl)-1-piperidinyl]-1-ethyl}-4-methoxy-1,3-dihydro-2H-indol-2-one

1-{2-[4-(4-Fluorobenzoyl)-1-piperidinyl]-1-ethyl}-3,3,5-trimethyl-1,3-dihydro-2H-indol-2-one

5-Chloro-3,3-dimethyl-1-{2-[4-(4-fluorobenzoyl)-1-piperidinyl]-1-ethyl}-1,3-dihydro-2H-indol-2-one

1-{2-[4-(4-Fluorobenzoyl)-1-piperidinyl]-1-ethyl}-3,3,7-trimethyl-1,3-dihydro-2H-indol-2-one

3,3-Dimethyl-1-{2-[4-(4-fluorobenzoyl)-1-piperidinyl]-1-ethyl}-5-methoxy-1,3-dihydro-2H-indol-2-one

1-{2-[4-(4-Fluorobenzoyl)-1-piperidinyl]-1-ethyl}-3,3,4-trimethyl-indol-2(3H)-one

1-{2-[4-(4-Fluorobenzoyl)-1-piperidinyl]-1-ethyl}-3,3,6-trimethyl-1,3-dihydro-2H-indol-2-one

1-{2-[4-(4-Fluorobenzoyl)-1-piperidinyl]-1-ethyl}-1,3-dihydro-2H-indol-2-one

1-{2-[4-(4-Fluorobenzoyl)-1-piperidinyl]-1-ethyl}-5-fluoro-1,3-dihydro-2H-indol-2-one

1-{2-[4-(4-Fluorobenzoyl)-1-piperidinyl]-1-ethyl}-3,3-difluoro-1,3-dihydro-2H-indole-2-one

1-{2-[4-(4-Fluorobenzoyl)-1-piperidinyl]-1-ethyl}-3,3,5-trifluoro-1,3-dihydro-2H-indole-2-one

1-(2-(4-(4-Fluorobenzoyl)-1-piperidinyl) -1-ethyl)-1,3-dihydro-3-spiro-1-cyclopropyl-2H-indole-2-one

1-(2-(4-(4-Fluorobenzoyl)-1-piperidinyl)-1-ethyl)-3-methyl-3-phenyl-1,3-dihydro-2H-indol-2-one

1-(2-(4-(4-Fluorobenzoyl)-1-piperidinyl)-1-ethyl)-1H-indol-2,3-dione monohydrochloride

The compounds of formula (I) can be prepared by methods described in European Patent Application 0 780 388.

Serotonin re-uptake inhibitors include fluoxetine, which is a preferred example.

Fluoxetine, N-methyl-3-(p-trifluoromethylphenoxy)-3-phenylpropylamine, is marketed in the hydrochloride salt form, and as the racemic mixture of its two enantiomers. U.S. Pat. No. 4,314,081 is an early reference on the compound. Robertson et al., J. Med. Chem. 31, 1412 (1988), taught the separation of the R and S enantiomers of fluoxetine and showed that their activity as serotonin uptake inhibitors is similar to each other. In this document, the word ‘fluoxetine’ will be used to mean any acid addition salt or the free base, and to include either the racemic mixture or either of the R and S enantiomers.

Duloxetine, N-methyl-3-(1-naphthalenyloxy)-3-(2-thienyl)propanamine, is usually administered as the hydrochloride salt and as the (+) enantiomer. It was first taught by U.S. Pat. No. 4,956,388, which shows its high potency. The word ‘duloxetine’ will be used here to refer to any acid addition salt or the free base of the molecule.

Venlafaxine is known in the literature, and its method of synthesis and its activity as an inhibitor of serotonin and norepinephrine uptake are taught by U.S. Pat. No. 4,761,501. Venlafaxine is identified as compound A in that patent.

Milnacipran (N,N-diethyl-2-aminomethyl-1-phenylcyclopropanecarboxamide) is taught by U.S. Pat. No. 4,478,836, which prepared milnacipran as its Example 4. The patent describes its compounds as antidepressants. Moret et al., Neuropharmacology 24, 1211-19 (1985), describe its pharmacological activities as an inhibitor of serotonin and norepinephrine reuptake;

Citalopram, 1-[3-dimethylamino)propyl]-1(4-fluorophenyl)-1,3-dihydro-5-isobenzofurancarbonitrile, is disclosed in U.S. Pat. No. 4,136,193 as a serotonin reuptake inhibitor. Its pharmacology was disclosed by Christensen et al., Eur. J. Pharmacol. 41, 153 (1977), and reports of its clinical effectiveness in depression may be found in Dufour et al., Int. Clin. Psychopharmacol. 2, 225 (1987), and Timmerman et al., ibid., 239;

Fluvoxamine, 5-methoxy-1-[4-trifluoromethyl)-phenyl]-1-pentanone 0-(2-aminoethyl)oxime, is taught by U.S. Pat. No. 4,085,225. Scientific articles about the drug have been published by Claassen et al., Brit. J. Pharmacol. 60, 505 (1977); and De Wilde et al., J. Affective Disord. 4, 249 (1982); and Benfield et al., Drugs 32, 313 (1986);

Paroxetine, trans-(−)-3-[(1,3-benzodioxol-5-yloxy)methyl]-4-(4-fluorophenyl)piperidine, may be found in U.S. Pat. Nos. 3,912,743 and 4,007,196. Reports of the drug's activity are in Lassen, Eur. J. Pharmacol. 47, 351 (1978); Hassan et al., Brit. J. Clin. Pharmacol. 19, 705 (1985); Laursen et al., Acta Psychiat. Scand. 71, 249 (1985); and Battegay et al., Neuropsychobiology 13, 31 (1985); and

Sertraline, (1S-cis)-4-(3,4-dichlorophenyl)-1,2,3,4-tetrahydro-N-methyl-1-naphthylamine hydrochloride, is a serotonin reuptake inhibitor which is marketed as an antidepressant. It is disclosed by U.S. Pat. No. 4,536,518.

As mentioned above, the compounds of formula (I) are antagonists at the serotonin, 5-HT_{1D} receptor. Their binding activity at the 5-HT_{1D} receptor has been demonstrated in a test described by Zgombick, J. M. et al, Molecular Pharmacology Vol. 40, 1992, pages 1036-1042.

Because of their ability to enhance the concentrations of 5-HT, the compositions of the present invention are indicated for use in treating a variety of conditions such as depression, obesity, bulimia, alcoholism, pain, hypertension, ageing, memory loss, sexual dysfunction, anxiety, schizophrenia, gastrointestinal disorders, headache, cardiovascular disorders, smoking cessation, drug addiction, emesis, epilepsy, Alzheimer's disease, obsessive compulsive disorder (OCD) and sleep disorders.

The compositions of the present invention may be particularly useful for the treatment of a condition selected from depression, anxiety, OCD and obesity.

The adjunctive therapy of the present invention is carried out by administering a first component together with the second component in any manner which provides effective levels of the compounds in the body at the same time. All of the compounds concerned are orally available and are normally administered orally, and so oral administration of the adjunctive combination is preferred. They may be administered together, in a single dosage form, or may be administered separately.

However, oral administration is not the only route or even the only preferred route. For example, transdermal administration may be very desirable for patients who are forgetful or petulant about taking oral medicine. One of the drugs may be administered by one route, such as oral, and the others may be administered by the transdermal, percutaneous, intravenous, intramuscular, intranasal or intrarectal route, in particular circumstances. The route of administration may be varied in any way, limited by the physical properties of the drugs and the convenience of the patient and the caregiver.

The adjunctive combination may be administered as a single pharmaceutical composition, and so pharmaceutical compositions incorporating both compounds are important embodiments of the present invention. Such compositions may take any physical form which is pharmaceutically acceptable, but orally usable pharmaceutical compositions are particularly preferred. Such adjunctive pharmaceutical compositions contain an effective amount of each of the compounds, which effective amount is related to the daily dose of the compounds to be administered. Each adjunctive dosage unit may contain the daily doses of all compounds, or may contain a fraction of the daily doses, such as one-third of the doses. Alternatively, each dosage unit may contain the entire dose of one of the compounds, and a fraction of the dose of the other compounds. In such case, the patient would daily take one of the combination dosage units, and one or more units containing only the other compounds. The amounts of each drug to be contained in each dosage unit depends on the identity of the drugs chosen for the therapy, and other factors such as the indication for which the adjunctive therapy is being given.

The inert ingredients and manner of formulation of the adjunctive pharmaceutical compositions are conventional, except for the presence of the combination of the present invention. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches and suspensions. In general, compositions contain from about 0.5% to about 50% of the compounds in total, depending on the desired doses and the types of compositions to be used. The amount of the compounds, however, is best defined as the effective amount, that is, the amount of each compound which provides the desired dose to the patient in need of such treatment. The activity of the adjunctive combinations do not depend on the nature of the composition, so the compositions are chosen and formulated solely for convenience and economy. Any of the combinations may be formulated in any desired form of composition. Some discussion of different compositions will be provided, followed by some typical formulations.

Capsules are prepared by mixing the compound with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such a sodium chloride and powdered sugar. Powdered cellulose derivatives are also useful.

Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylycellulose and waxes can also serve as binders.

A lubricant is necessary in a tablet formulation to prevent the tablet and punches from sticking in the die. The lubricant is chosen from such slippery solids as talc, magnesium and calcium stearate, stearic acid and hydrogenated vegetable oils.

Tablet disintegrators are substances which swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethylcellulose, for example, may be used, as well as sodium lauryl sulfate.

Enteric formulations are often used to protect an active ingredient from the strongly acid contents of the stomach. Such formulations are created by coating a solid dosage form with a film of a polymer which is insoluble in acid environments, and soluble in basic environments. Exemplary films are cellulose acetate phthalate, polyvinyl acetate phthalate, hydroxypropyl methylcellulose phthalate and hydroxypropyl methylcellulose acetate succinate.

Tablets are often coated with sugar as a flavour and sealant. The compounds may also be formulated as chewable tablets, by using large amounts of pleasant-tasting substances such as mannitol in the formulation, as is now well-established practice. Instantly dissolving tablet-like formulations are also now frequently used to assure that the patient consumes the dosage form, and to avoid the difficulty in swallowing solid objects that bothers some patients.

When it is desired to administer the combination as a suppository, the usual bases may be used. Cocoa butter is a traditional suppository base, which may be modified by addition of waxes to raise its melting point slightly. Water-miscible suppository bases comprising, particularly, polyethylene glycols of various molecular weights are in wide use, also.

Transdermal patches have become popular recently. Typically they comprise a resinous composition in which the drugs will dissolve, or partially dissolve, which is held in contact with the skin by a film which protects the composition. Many patents have appeared in the file recently. Other, more complicated patch compositions are also in use, particularly those having a membrane pierced with innumerable pores through which the drugs are pumped by osmotic action.

The following formulations illustrate the invention:

### Formulation 1

Hard gelatin capsules are prepared using the following ingredients:

| | Quantity | |
|---|---|---|
| | (mg/capsule) | |
| Fluoxetine, racemic, hydrochloride | 20 | |
| Compound of formula (I) | 30 | |
| Starch, dried | 200 | |
| Magnesium stearate | 10 | |
| Total | 260 | mg |

### Formulation 2

A tablet is prepared using the ingredients below:

| | Quantity | |
|---|---|---|
| | (mg/capsule) | |
| Fluoxetine, racemic, hydrochloride | 10 | |
| Compound of formula (I) | 40 | |
| Cellulose, microcrystalline | 400 | |
| Silicon dioxide, fumed | 10 | |
| Stearic acid | 5 | |
| Total | 465 | mg |

The components are blended and compressed to form tablets each weighing 465 mg.

### Formulation 3

An aerosol solution is prepared containing the following components:

| | Weight |
|---|---|
| (+)-Duloxetine, hydrochloride | 10 |
| Compound of formula (I) | 10 |
| Ethanol | 25.75 |
| Propellant 22 (Chlorodifluoromethane) | 70 |
| Total | 115.75 |

The active compound is mixed with ethanol and the mixture added to a portion of the propellant 22, cooled to −30 C. and transferred to a filling device. The required amount is then fed to a stainless steel container and diluted with the remainder of the propellant. The valve units are then fitted to the container.

### Formulation 4

Suppositories, each containing 45 mg of active ingredient, are made as follows:

| | | |
|---|---|---|
| (+)-Duloxetine, hydrochloride | 5 | |
| Compound of formula (I) | 40 | |
| Saturated fatty acid glycerides | 2,000 | |
| Total | 2,045 | mg |

The active ingredient is passed through a No. 60 mesh U.S. sieve and suspended in the saturated fatty acid glycerides previously melted using the minimum heat necessary. The mixture is then poured into a suppository mould of nominal 2 g capacity and allowed to cool.

### Formulation 5

Suspensions, each containing 70 mg of active ingredient per 5 ml dose, are made as follows:

| Fluoxetine, racemic, hydrochloride | 10 | mg |
|---|---|---|
| Compound of formula (I) | 60 | mg |
| Sodium carboxymethyl cellulose | 50 | mg |
| Syrup | 1.25 | ml |
| Benzoic acid solution | 0.10 | ml |
| Flavour | q.v. | |
| Colour | q.v. | |
| Purified water to total | 5 | ml |

The active ingredient is passed through a No. 45 mesh U.S. sieve and mixed with the sodium carboxymethyl cellulose and syrup to form a smooth paste. The benzoic acid solution, flavour and colour are diluted with a portion of the water and added, with stirring. Sufficient water is then added to produce the required volume.

### EXAMPLE

Acute Administration

Animals with dialysis probes inserted in brain regions were placed in the dialysis apparatus and the probes perfused with artificial cerebrospinal fluid. Samples of perfusate were collected every 20 minutes after an initial 30 minute washout period. Basal samples were collected for 2 hours before administration of drug or vehicle by the oral route. Further samples were collected for up to 4 hours post drug administration. Samples were analysed for 5-HT by HPLC with electrochemical detection.

Data was expressed as a percentage of a pre-injection control, obtained by averaging the last three samples prior to drug/vehicle administration. Transformed data (natural log of percentages) was analysed by ANOVA with repeated measures.

Chronic Administration

The method differed from that for acute administration in that animals were treated with fluoxetine or vehicle for 21 days prior to placement in the dialysis apparatus. The subsequent manipulations were as described above. The 5-HT concentration in the treated group was expressed as the percentage increase over vehicle treated controls.

TABLE

| | % increase in |
|---|---|
| Treatment | 5-HT concentrations |
| Acute Fluoxetine at 10 or 20 | 99 |
| mg/kg (maximum possible response) | |
| Chronic (21 day) Fluoxetine at | 184 |
| 10 mg/kg | |
| Acute Compound of formula (I)* at | 110 |
| 10 mg/kg | |
| Acute Fluoxetine at 20 mg/kg plus | 460 |
| Acute Compound of formula (I)* at | |
| 10 mg/kg | |

*compound of formula (I) 1-[2-[4-(4-fluorobenzoyl)-1-piperidinyl]ethyl]-1,3-dihydro-3,3-dimethyl-2H-indol-2-one, monohydrochloride.

Thus the combination of acute fluoxetine and acute compound of formula (I), a 5-HT_{1D} antagonist, is able to elevate 5-HT concentrations to a level greater than can be achieved by the acute administration of SSRI. The level achieved is at least as great as that obtained by the chronic administration of an SSRI. It is well documented that SSRI's are only effective in the treatment of depression after chronic administration. The data indicates that the combination of a compound of formula (I) and an SSRI will produce early onset antidepressant activity in the clinic.

1. A pharmaceutical composition comprising an inhibitor of the serotonin transporter and a compound of the formula: 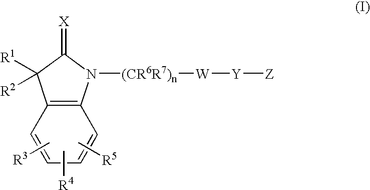
in which
R¹ and R² are each hydrogen, C₁₋₄ alkyl, C₁₋₄ alkoxy, HO—C₁₋₄ alkyl, C₁₋₄ alkoxy-C₁₋₄ alkyl, C₁₋₄ alkylthio, halo, Ph, PhCR′R″— where Ph is optionally substituted phenyl and R′ and R″ are each hydrogen or C₁₋₄ alkyl, or R¹ and R² together with the carbon atom to which they are attached form a C₃₋₆ cycloalkyl group, >C═O, >C═NOR′ where R′ is hydrogen or C₁₋₄ alkyl,
R³, R⁴ and R⁵ are each hydrogen, halo, nitro, C₁₋₄ alkyl, C₁₋₄ alkoxy, C₁₋₄ alkylthio, C₁₋₄ alkyl-CO—, C₁₋₄ alkyl-S(O)ₘ— where m is 0, 1 or 2, R′R″N—SO₂—, —COOR′, —CONR′R″, —NR′R″, —N(OR′)COOR″, —COR′,
—NHSO₂R′, where R′ and R″ are each hydrogen or C₁₋₄ alkyl,
R⁶ and R⁷ are each hydrogen or C₁₋₄ alkyl, and n is 1 to 6,
X is oxygen or sulphur,
W is 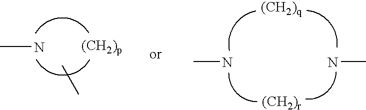
where p is 4 to 7, and q and r are each 1 to 3,
Y is 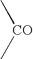
or —CH(OH)—,
and
Z is optionally substituted phenyl or optionally substituted heteroaryl;
or a pharmaceutically acceptable salt or ester thereof;
together with a pharmaceutically acceptable diluent or carrier therefor.

2. A pharmaceutical composition according to claim 1, which comprises a compound of the formula 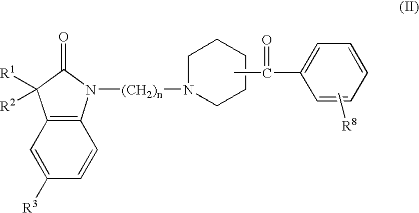
and preferably one in which R¹ and R² are each hydrogen or C₁₋₄ alkyl, R³ is hydrogen, C₁₋₄ alkyl or halo, n is 2, and R⁸ is hydrogen or halo. Preferably the benzoyl substituent is attached to the piperidinyl ring at the 4-position. A particularly preferred group is one in which R¹ and R² are both hydrogen and R³ is hydrogen or fluoro, n is 2 and R⁸ is halo, preferably fluoro; a pharmaceutically acceptable salt thereof.

3. A pharmaceutical composition according to any of claims 1 and 2, in which the serotonin transport inhibitor is fluoxetine, duloxetine, venlafaxine, milnacipran, citalopram, fluovoxamine, sertraline or nefazodone.

4. A pharmaceutical composition according to claim 3, in which the serotonin transport inhibitor is fluoxetine.

5. A pharmaceutical composition according to claim 4, in which the serotonin transport inhibitor is R-fluoxetine.

6. A method for treating a patient suffering from or susceptible to a disorder of the central nervous system, which comprises administering a combination of a serotonin transport inhibitor and a 5-HT_{1D} antagonist selected from a compound of formula I as defined in claim 1 and a compound of formula II as defined in claim 2.

7. A combination of an inhibitor of the serotonin transporter and a 5HT_{1D} antagonist selected from a compound of formula I as defined in claim 1 and a compound of formula II as defined in claim 2, for use as a medicament.

8. The use of a combination of an inhibitor of the serotonin transporter and a 5HT1D antagonist selected from a compound of formula I as defined in claim 1 and a compound of formula II as defined in claim 2, for the manufacture of a medicament for the treatment of a disorder of the central nervous system.

9. The use of a combination of an inhibitor of the serotonin transporter and a 5HT_{1D} antagonist selected from a compound of formula I as defined in claim 1 and a compound of formula II as defined in claim 2, for the manufacture of a medicament for the treatment of a disorder selected from depression, anxiety, OCD and depression.

10. The use of a combination of an inhibitor of the serotonin transporter and 5HT_{1D} antagonist selected from a compound of formula I as defined in claim 1 and a compound of formula II as defined in claim 2, for the manufacture of a medicament for enhancing the action of a selective serotonin re-uptake inhibitor in increasing the availability of serotonin.
